(19) United States
(12) Patent Application Publication
(10) Pub. No.: US 2014/0376685 A1
(43) Pub. Date: Dec. 25, 2014

(54) METHOD FOR 3D MINERAL MAPPING OF A ROCK SAMPLE

(71) Applicants: Dmitry Anatolievich Koroteev, Moscow (RU); Albina Rishatovna Mutina, Bogota (CO); Alexander Nikolaevich Nadeev, Spring, TX (US); Omer M. Gurpinar, Denver, CO (US)

(72) Inventors: Dmitry Anatolievich Koroteev, Moscow (RU); Albina Rishatovna Mutina, Bogota (CO); Alexander Nikolaevich Nadeev, Spring, TX (US); Omer M. Gurpinar, Denver, CO (US)

(73) Assignee: SCHLUMBERGER TECHNOLOGY CORPORATION, SUGAR LAND, TX (US)

(21) Appl. No.: 14/352,544

(22) PCT Filed: Oct. 18, 2011
(86) PCT No.: PCT/RU2011/000813

§ 371 Date: Sep. 4, 2014

(51) Int. Cl. G01N 23/04; G01N 23/223; G01N 33/24; G01N 23/207

(52) U.S. Cl. CPC G01N 23/046; G01N 23/2076; G01N 23/223; G01N 33/24
USPC 378/4

(57) ABSTRACT

The method for 3D mineral mapping of a rock sample comprises the steps of defining a total mineral content of a sample and calculating X-ray attenuation coefficients for the defined minerals. X-ray micro/nanoCT scanning of the sample is performed and its three-dimensional microstructure image in gray scale is obtained. Characteristic grayscale levels in the image corresponding to calculated X-ray attenuation coefficients and accordingly to the minerals are allocated and the 3D mineral map of the interior of the sample is provided.

### FIELD OF THE INVENTION

The invention relates to X-ray based analysis of a core sample, namely microtomography (microCT) and nanotomography (nanoCT) techniques.

### BACKGROUND OF THE INVENTION

X-ray micro- and nano-computed tomography is a well-known non-destructive technique for visualizing and quantifying the internal structure of objects in three dimensions (3D). It is used to provide high resolution images of rocks in 2D or 3D at a micron scale (see, for example, M. A. Knackstedt et al., “Digital Core Laboratory: Properties of Reservoir Core Derived From 3D Images,” SPE 87009, 2004).

The idea of linking the gray values in 3D microCT image with densities is reflected in United States Patent Application Publication Number 2005/0010106. However, the application does not disclose any procedures dealing with location of the objects with different densities and chemical composition, such as different mineral grains.

### SUMMARY OF THE INVENTION

A method that allows providing an adequate geometry of a pore space and to perform mapping of minerals inside a sample is proposed. The mineral mapping in 3D allows saving the information about rock wetting and elastic properties inside the core sample.

The method for 3D mineral mapping of a rock sample comprises the steps of defining a total mineral content of a sample and calculating X-ray attenuation coefficients for the defined minerals. X-ray micro/nanoCT scanning of the sample is performed and its three-dimensional microstructure image in gray scale is obtained. Characteristic grayscale levels in the image corresponding to calculated X-ray attenuation coefficients and accordingly to the minerals are allocated and the 3D mineral map of the interior of the sample is provided.

Characteristic sizes of the mineral grains can be defined and then minerals with the grains having characteristic size higher than resolution limit of micro/nanoCT scanning mode are selected.

Total mineral content for the sample is defined using one of the conventional mineralogy characterization method: thin section petrography analysis, X-ray fluorescence (XRF), powder/single cristal X-ray diffraction (XRD), Confocal Raman microscopy.

Characteristic sizes of mineral grains might be estimated from petrography analysis, BET method, Confocal Raman/Raman spectroscopy or Atomic Force microscopy.

As the proposed method is applicable for 3D image of a geological sample obtain by X-ray CT technique with both micro and nano resolution, we will further refer to X-ray microCT example.

### BRIEF DESCRIPTION OF THE DRAWINGS

FIG. 1a represents a vertical cross-section of 3D microCT image in grayscale;

FIG. 1b represents volume rendering of segmented 3D microCT image;

FIG. 1c shows a distribution of the minerals along the vertical axis of the test tube.

### DETAILED DESCRIPTION OF THE INVENTION

Microtomography (microCT) and nanotomography (nanoCT) techniques can be used for adequate petrophysical characterization of the core sample via numerical modeling of the monophase and multiphase flows in pores of the sample and for accurate numerical characterization of thermal, electrical, wettability and geomechanical properties of the rock sample. These rock properties are essential for oil/gas reservoir exploration and management. The three dimensional (3D) mineral mapping can also find multiple applications in characterizing the rocks with non-hydrocarbon mineral resources (like rocks with coal or/and different metals), for instance, in the mining industry.

Different minerals have different chemical content (chemical elements) and density. In the other words, the minerals possess different contras in X-ray back projection and might be differentiated by their X-ray absorption coefficients:

I=I₀e^{-μl}

I—X-ray radiation intensity after l length propagation, I₀—initial X-ray radiation intensity, μ—linear attenuation coefficient (cm⁻¹), l—length of radiation propagation (cm). This feature allows expecting different grayscale levels which correspond to volumes occupied by grains of different minerals in 3D micro/nanoCT image of a rock sample. Having known what minerals (M1, M2, . . . , Mn) dominate in the sample, it is possible to estimate the values of X-ray attenuation coefficient for them (K1, K2, . . . , Kn).

The method comprises the following steps. At first, the data for total mineral content for the sample is collected using one of the known methods—petrography, X-ray fluorescence (XRF), powder/single cristal X-ray diffraction (XRD), Confocal Raman/Raman spectroscopy or Scanning electron imaging (see, for example, Petrographic thin section analysis—www.ncptt.nps.gov/digital-image-analysis-of-petrographic-thin-sections-in-conservation-research-2004-01/; micro-XRF analysis—www.horiba.com/fileadmin/uploads/Scientific/Documents/XRay/xgtmin01.pdf; XRD analysis—Ore Geology Reviews, Volume 6, Issues 2-3, May 1991, Pages 107-118, Applied Mineralogy in Exploration).

Then, characteristic sizes of the mineral grains can be defined; characteristic sizes might be estimated from Petrography, Confocal Raman/Raman spectroscopy or Scanning electron imaging (Raman microscopic imaging (see, for example http://www.witec-instruments.de/en/download/Raman/Geoscience.pdf or http://www.fei.com/applications/industry/).

Petrography (optical mineralogy) is the study of minerals and rocks by measuring their optical properties. Most commonly, rock and mineral samples are prepared as thin sections or grain mounts for study in the laboratory with a petrographic microscope. Optical mineralogy is used to identify the mineralogical composition of geological materials in order to help reveal their origin and evolution (see www.ncptt.nps.gov/digital-image-analysis-of-petrographic-thin-sections-in-conservation-research-2004-01).

X-ray fluorescence (XRF) is the emission of characteristic “secondary” (or fluorescent) X-rays from a material that has been excited by bombarding with high-energy X-rays or gamma rays. The phenomenon is widely used for elemental analysis and chemical analysis, particularly in the investigation of metals, glass, ceramics and building materials, and for research in geochemistry, forensic science and archaeology. The example of XRF application for geosamples can be found here: http://www.horiba.com/fileadmin/uploads/Scientific/Documents/XRay/xgtmin01.pdf

The Confocal Raman microscopes record a Raman spectra at each pixel of 2D area of a sample within a field of view. Decoding the spectra gives the chemical compound in the pixel. In case of natural rocks, areas with same chemical compounds are then assigned to different minerals (see http://www.witec-instruments.de/en/download/Raman/Geoscience.pdf).

X-ray diffraction yields the atomic structure of materials and is based on the elastic scattering of X-rays from the electron clouds of the individual atoms in the system. The most comprehensive description of scattering from crystals is given by the dynamical theory of diffraction. Powder diffraction (XRD) is a technique used to characterise the crystallographic structure, crystallite size (grain size), and preferred orientation in polycrystalline or powdered solid samples. Powder diffraction is commonly used to identify unknown substances, by comparing diffraction data against a database maintained by the International Centre for Diffraction Data (XRD analysis—Ore Geology Reviews, Volume 6, Issues 2-3, May 1991, Pages 107-118, Applied Mineralogy in Exploration).

Energy-dispersive X-ray spectroscopy (EDX) is an analytical technique used for the elemental analysis or chemical characterization of a sample. It is one of the variants of X-ray fluorescence spectroscopy which relies on the investigation of a sample through interactions between electromagnetic radiation and matter, analyzing X-rays emitted by the matter in response to being hit with charged particles. Its characterization capabilities are due in large part to the fundamental principle that each element has a unique atomic structure allowing X-rays that are characteristic of an element's atomic structure to be identified uniquely from one another. EDX systems are most commonly found on scanning electron microscopes (SEM-EDX) and electron microprobes. Scanning electron microscopes are equipped with a cathode and magnetic lenses to create and focus a beam of electrons, and since the 1960s they have been equipped with elemental analysis capabilities. A detector is used to convert X-ray energy into voltage signals; this information is sent to a pulse processor, which measures the signals and passes them onto an analyzer for data display and analysis (http://www.fei.com/applications/industry/)

The minerals (M1, M2, . . . , Mn) which have grains (which fill areas) with characteristic size higher than resolution limit are selected. The resolution of micro/nanoCT scanning mode which is planned for tomographic experiment is defined using data on sample size and technical description of micro/nanoCT setup.

X-ray attenuation coefficients (K1, K2, . . . , Kn) for all defined minerals or selected minerals (M1, M2, . . . , Mn) having grains with characteristic size higher than resolution limit are calculated. The estimation for X-ray attenuation coefficients might be performed using NIST database, for example (http://www.nist.gov/pml/data/xraycoef/index.cfm).

High resolution micro/nanoCT experiment with the sample is performed and the 3D micro/nanoCT image in gray scale is obtained.

Then, n characteristic grayscale levels in the image with average grayscale values L1, L2, . . . , Ln (L1<L2< . . . <Ln) corresponding to K1, K2, . . . , Kn are allocated. The procedure might be performed either automatically, using any of image segmentation methods (see, for example, http://dspace.nitle.org/bitstream/handle/10090/781/s10csci2007blasiak.pdf?sequence=1), or manually by choosing appropriate thresholds at image graylevel histogram.

The allocation might be forewarned by pre-processing of the 3D image by one of the edge-preserving image filters (see for example http://math.nist.gov/mcsd/savg/software/filters/smooth/index.html)

The micro/nanoCT image is represented in a segmented form, at which areas of characteristic gray levels correspond to particular minerals, i.e. the 3D mineral map of the interior of the sample is built. Brightest areas correspond to a mineral with the highest value of X-ray attenuation coefficient, darkest areas—to minerals having lowest X-ray attenuation, and minerals with medium X-ray attenuations have medium grayscale intensity.

The methodology was tested in lab. The powder, containing granules of three pure minerals was prepared. The minerals were: Quartz, Halite, and Calcite. SkyScan 1172 microCT technique was used for tomographic experiment. The grain sizes were much higher than resolution limit of scanning mode used in tomographic experiment. The estimations for X-ray attenuation for the minerals gave the following: 2.3 cm⁻¹ for quartz, 3.9 cm⁻¹ for halite, and 5.0 cm⁻¹ for calcite. The granules were placed in a test tube in the following order (from bottom to top): quartz, halite, and calcite. The test tube was placed in the microCT technique for tomographic experiment.

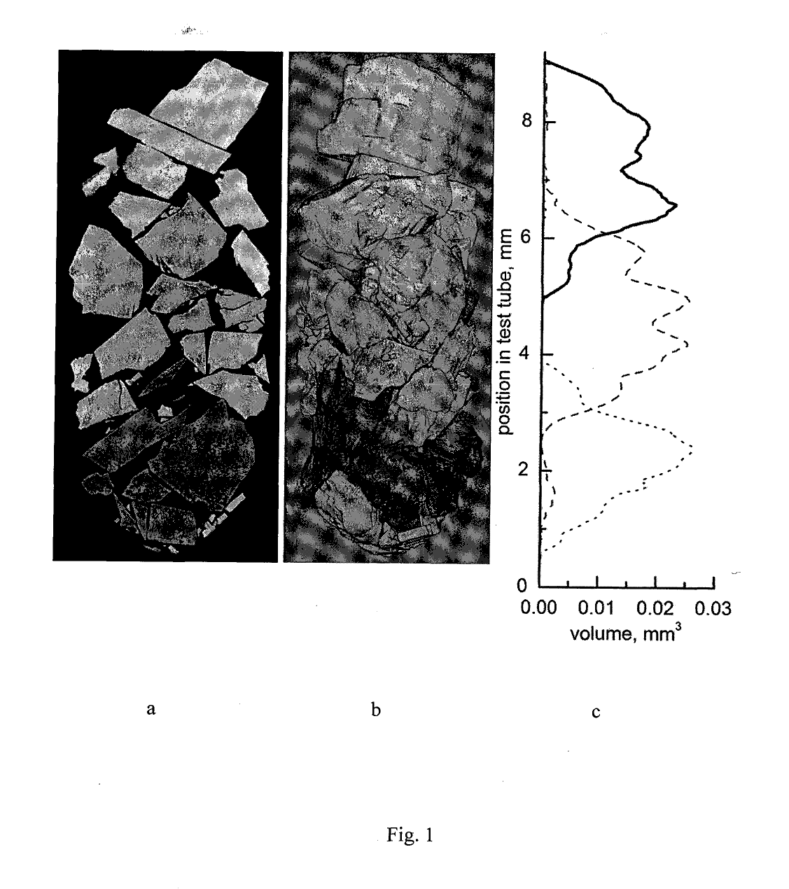

FIG. 1a represents a vertical cross-section of 3D microCT image in grayscale. The image was acquired at X-ray microCT experiment at pixel size resolution of 2.5 micron. Brightest areas at the top zone correspond to calcite—a mineral with the highest value of X-ray attenuation coefficient from ones considered in experiment. Darkest areas in the bottom zone stand for quartz which has lowest X-ray attenuation. In the middle there are some halite granules having medium grayscale intensity and medium X-ray attenuations.

FIG. 1b represents volume rendering of segmented 3D microCT image. In the other words, FIG. 1b visualizes 3D mineral map. Segmentation was performed by simple thresholdings in the local minima of the histogram of 3D microCT image. I.e. graylevels between neighboring local minima were assigned to particular minerals. This simple option was enabled because the histogram of the microCT image represents clear three-peak curve.

FIG. 1c shows a distribution of the minerals along the vertical axis of the test tube (volume, occupied by a mineral VS vertical position in test tube). The distribution was calculated using 3D map represented in FIG. 1b. Solid line represents calcite, dashed line represents halite, dotted line represents quartz.

1. A method for 3D mineral mapping of a rock sample, comprising:
defining a total mineral content for the sample;
calculating X-ray attenuation coefficients for minerals defined in the total mineral content;
performing X-ray micro/nanoCT scanning of the sample;
providing a 3D mineral map of the sample.

2. A method of claim 1 wherein characteristic sizes of the mineral grains are defined and minerals with grains having a characteristic size higher than a resolution limit of a micro/nanoCT scanning mode are selected.

3. A method of claim 1 wherein mineral content of the sample is defined by petrography analysis.

4. A method of claim 1 wherein mineral content of the sample is defined by X-ray fluorescence.

5. A method of claim 1 wherein mineral content of the sample is defined by X-ray diffraction.

6. A method of claim 1 wherein mineral content of the sample is defined by Confocal Raman imaging/Raman spectroscopy.

7. A method of claim 1 wherein mineral content of the sample is defined by Scanning electron imaging.

8. A method of claim 2 wherein characteristic sizes of the mineral grains are defined from petrography.

9. A method of claim 2 wherein characteristic sizes of the mineral grains are defined from Confocal Raman imaging/Raman spectroscopy.

10. A method of claim 1 wherein before allocating characteristic grayscale the 3D micro/nanoCT image is processed with an edge-preserving image filter.

11. An apparatus for providing a three dimensional mineral map of a sample, comprising:
a machine to define total mineral content of the sample, wherein the sample comprises mineral grains;
an X-ray micro CT scanning machine, nanoCT scanning machine, or both; and
a device to calculate X-ray attenuation coefficients for minerals defined in the total mineral content and to provide a 3D mineral map of the sample.

12. An apparatus of claim 11 further comprising a machine to measure grain size.

13. An apparatus of claim 11 wherein a machine to define total mineral content of the sample comprises a device for performing petrography analysis.

14. An apparatus of claim 11 wherein a machine to define total mineral content of the sample comprises a device for performing X-ray fluorescence.

15. An apparatus of claim 11 wherein a machine to define total mineral content of the sample comprises a device for performing X-ray diffraction.

16. An apparatus of claim 11 wherein a machine to define total mineral content of the sample comprises a device for performing scanning electron imaging.

17. Apparatus of claim 11 wherein a machine to define total mineral content of the sample comprises a device for performing Confocal Raman imaging/Raman spectroscopy.

18. Apparatus of claim 12 wherein a machine to measure grain size comprises a device for performing petrography analysis.

19. Apparatus of claim 12 wherein a machine to measure grain size comprises a device for performing Confocal Raman imaging/Raman spectroscopy.

20. Apparatus of claim 11 further comprising an edge-preserving image filter.
